(19) United States
(12) Patent Application Publication
(10) Pub. No.: US 2024/0193414 A1
(43) Pub. Date: Jun. 13, 2024

(54) PHYSICS AWARE TRAINING FOR DEEP PHYSICAL NEURAL NETWORKS

(71) Applicant: NTT RESEARCH, INC., Sunnyvale, CA (US)

(72) Inventors: Logan G. WRIGHT, Sunnyvale, CA (US); Tatsuhiro ONODERA, Sunnyvale, CA (US); Peter L. MCMAHON, Ithaca, NY (US); Martin STEIN, Ithaca, NY (US)

(73) Assignee: NTT RESEARCH, INC., Sunnyvale, CA (US)

(21) Appl. No.: 18/287,808

(22) PCT Filed: Apr. 21, 2022
(86) PCT No.: PCT/US2022/025830

§ 371 Date: Oct. 20, 2023

Related U.S. Application Data
(60) Provisional application No. 63/178,318, filed on Apr. 22, 2021.

(51) Int. Cl. G06N 3/08

(52) U.S. Cl. CPC G06N 3/08

(57) ABSTRACT

A physical neural network system includes a physical and digital component. The digital component includes a computing system. The physical component and the digital component work in conjunction to execute a physics aware training process. The physics aware training process includes generating, by the digital component, an input data set for input to the physical component, applying, by the physical component, one or more transformations to the input data set to generate an output for a forward pass of the physics aware training process, comparing, by the digital component, the generated output to a canonical output to determine an error, generating, by the digital component, a loss gradient using a differentiable digital model for a backward pass of the physics aware training process, and updating, by the digital component, training parameters for subsequent input to the physical component based on the loss gradient.

### CROSS-REFERENCE TO RELATED APPLICATIONS

This application claims priority to U.S. Provisional Application Ser. No. 63/178,318, filed Apr. 22, 2021, which is hereby incorporated by reference in its entirety.

### FIELD OF DISCLOSURE

The present disclosure generally relates to deep physical neural networks and, in particular, to deep physical neural networks trained using a backpropagation method for arbitrary physical systems.

### BACKGROUND

Deep neural networks are growing in their applications across business, technology, and science. As deep neural networks continue to grow in scale, so too does the energy that these deep neural networks consume. While the hardware sphere was initially able to keep pace with the developments in deep neural network technology, the advances in deep learning are occurring so quickly that they are outpacing Moore's law.

### SUMMARY

In some embodiments, a physical neural network system is disclosed herein. The physical neural network system includes a physical component a digital component. The digital component includes a computing system. The physical component and the digital component work in conjunction to execute a physics aware training process. The physics aware training process includes generating, by the digital component, an input data set for input to the physical component. The physics aware training process further includes applying, by the physical component, one or more transformations to the input data set to generate an output for a forward pass of the physics aware training process. The physics aware training process further includes, based on the generated output, comparing, by the digital component, the generated output to a canonical output to determine an error. The physics aware training process further includes generating, by the digital component, a loss gradient using a differentiable digital model for a backward pass of the physics aware training process. The physics aware training process further includes updating, by the digital component, training parameters for subsequent input to the physical component based on the loss gradient.

In some embodiments, a method of training a physical neural network is disclosed herein. A digital component of the physical neural network generates an input data set for input to the physical component. A physical component of the physical neural network applies one or more transformations to the input data set to generate an output for a forward pass of the training. Based on the generated output, the digital component compares the generated output to a canonical output to determine an error. The digital component generates a loss gradient using a differentiable digital model for a backward pass of the training. The digital component updates training parameters for subsequent input to the physical component based on the loss gradient.

In some embodiments, a non-transitory computer readable medium is disclosed herein. The non-transitory computer readable medium includes one or more sequences of instructions, which, when executed by one or more processors, causes a computing system to perform operations. The operations include generating an input data set for input to a physical component of a physical neural network. The operations further include causing the physical component of the physical neural network to apply one or more transformations to the input data set to generate an output for a forward pass of a training process. The operations further include based on the generated output, comparing the generated output to a canonical output to determine an error. The operations further include generating a loss gradient using a differentiable digital model for a backward pass of the training process. The operations further include updating training parameters for subsequent input to the physical component based on the loss gradient.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrated only typical embodiments of this disclosure and are therefore not to be considered limiting of its scope, for the disclosure may admit to other equally effective embodiments.

FIG. 1A is a block diagram illustrating an exemplary physical neural network, according to example embodiments.

FIG. 1B is a block diagram illustrating physical neural network in more detail, according to example embodiments.

FIG. 2 is a block diagram illustrating a generic physical neural network system, according to example embodiments.

FIG. 3A is a block diagram illustrating a conventional backpropagation process, according to example embodiments.

FIG. 3B is a block diagram illustrating a physics-aware backpropagation process, according to example embodiments.

FIG. 4 is a block diagram illustrating a detailed physics-aware training process, according to example embodiments.

FIG. 5 is a flow diagram illustrating a method of training a physical neural network using physics-aware training, according to example embodiments.

FIG. 6 is a block diagram illustrating an example physical neural network, according to example embodiments.

FIG. 7A illustrates a system bus architecture of computing system, according to example embodiments.

FIG. 7B illustrates a computer system having a chipset architecture, according to example embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Deep neural networks have become a pervasive tool in science and engineering. However, the growing energy requirements of modern deep neural networks increasingly limit their scaling and broader use. To account for this, one or more techniques described herein propose a radical alternative for implementing deep neural network models through physical neural networks. For example, disclosed herein is a hybrid physical-digital algorithm referred to as “Physics-Aware Training” that efficiently trains sequences of controllable physical systems to act as deep neural networks. Such approach automatically trains the functionality of any sequence of real physical systems, directly, using backpropagation, the same technique used for modern deep neural networks. Physical neural networks may facilitate unconventional machine learning hardware that is orders of magnitude faster and more energy efficient than conventional electronic processors.

Like many historical developments in artificial intelligence the widespread adoption of deep neural networks (DNNs) was enabled in part by synergistic hardware. In 2012, building on numerous earlier works, Krizhevsky et al. showed that the backpropagation algorithm for stochastic gradient descent (SGD) could be efficiently executed with graphics-processing units to train large convolutional DNNs to perform accurate image classification. Since 2012, the breadth of applications of DNNs has expanded, but so too has their typical size. As a result, the computational requirements of DNN models have grown rapidly, outpacing Moore's Law. Now, DNNs are increasingly limited by hardware energy efficiency.

The emerging DNN energy problem has inspired special-purpose hardware: DNN “accelerators”. Several proposals push beyond conventional electronics with alternative physical platforms, such as optics or memristor crossbar arrays. These devices typically rely on approximate analogies between the hardware physics and the mathematical operations in DNNs. Consequently, their success will depend on intensive engineering to push device performance toward the limits of the hardware physics, while carefully suppressing parts of the physics that violate the analogy, such as unintended nonlinearities, noise processes, and device variations.

More generally, however, the controlled evolutions of physical systems are well-suited to realizing deep learning models. DNNs and physical processes share numerous structural similarities, such as hierarchy, approximate symmetries, redundancy, and nonlinearity. These structural commonalities explain much of DNNs' success operating robustly on data from the natural, physical world. As physical systems evolve, they perform, in effect, the mathematical operations within DNNs: controlled convolutions, nonlinearities, matrix-vector operations and so on. These physical computations can be harnessed by encoding input data into the initial conditions of the physical system, then reading out the results by performing measurements after the system evolves. Physical computations can be controlled by adjusting physical parameters. By cascading such controlled physical input-output transformations, trainable, hierarchical physical computations can be realized. As anyone who has simulated the evolution of complex physical systems appreciates, physical transformations are typically faster and consume less energy than their digital emulations: processes which take nanoseconds and nanojoules frequently require seconds and joules to digitally simulate. PNNs are therefore a route to scalable, energy-efficient, and high-speed machine learning.

Theoretical proposals for physical learning hardware have recently emerged in various fields, such as optics, spintronic nano-oscillators, nanoelectronic devices, and small-scale quantum computers. A related trend is physical reservoir computing, in which the information transformations of a physical system ‘reservoir’ are not trained but are instead linearly combined by a trainable output layer. Reservoir computing harnesses generic physical processes for computation, but its training is inherently shallow: it does not allow the hierarchical process learning that characterizes modern deep neural networks. In contrast, the newer proposals for physical learning hardware overcome this by training the physical transformations themselves.

There have been few experimental studies on physical learning hardware, however, and those that exist have relied on gradient-free learning algorithms. While these works have made critical steps, it is now appreciated that gradient-based learning algorithms, such as the backpropagation algorithm, are essential for the efficient training and good generalization of large-scale DNNs. To solve this problem, proposals to realize backpropagation on physical hardware have appeared. While inspirational, these proposals nonetheless often rely on restrictive assumptions, such as linearity or dissipation-free evolution. The most general proposals may overcome such constraints, but still rely on performing training in silico, i.e., wholly within numerical simulations. Thus, to be realized experimentally, and in scalable hardware, they will face the same challenges as hardware based on mathematical analogies: intense engineering efforts to force hardware to precisely match idealized simulations.

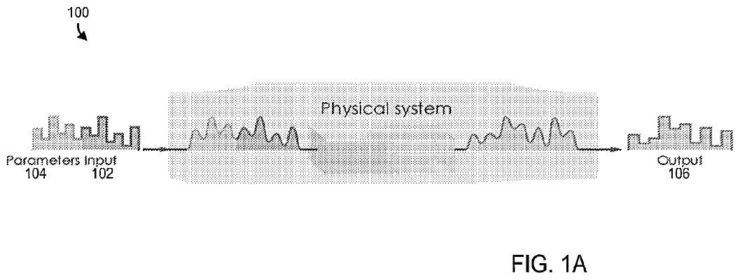

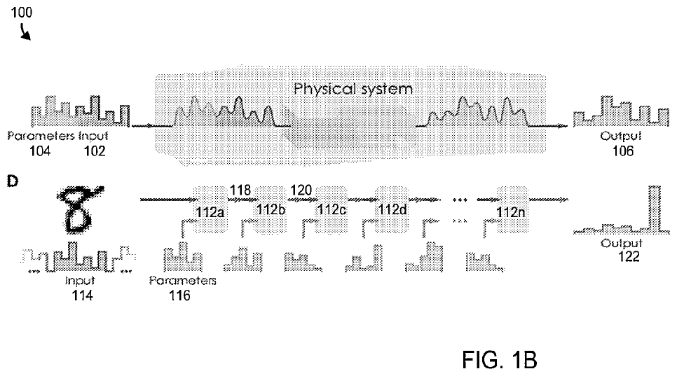

FIG. 1A is a block diagram illustrating an exemplary physical neural network 100, according to example embodiments. To facilitate discussion of physical neural network 100, the following is a short discussion of conventional artificial neural networks. Conventional artificial neural networks (ANN) are typically built from an operational unit (the layer) that involves a trainable matrix-vector multiplication followed by element-wise nonlinear activation such as the rectifier (ReLU). The weights of the matrix-vector multiplication may be adjusted during training so that the ANN implements a desired mathematical operation. A deep neural network (DNN) may be created by cascading these operational units together. The DNN may be trained to learn a multi-step (hierarchical) computation. When physical systems evolve, they may perform computations. The controllable properties of the system may be partitioned into input data and control parameters. By changing control parameters, the physical transformation performed on the input data can be altered.

As shown in FIG. 1A, physical neural network 100 may receive, as input, data input 102 and parameters 104. Based on data input 102 and parameters 104, physical neural network 100 may generate output 106. Just as hierarchical information processing is realized in DNNs by a sequence of trainable nonlinear functions, deep physical neural networks 100 can be created by cascading layers of trainable physical transformations. In physical neural network 100, each physical layer implements a controllable function, which can be of a more general form than that of a conventional DNN layer.

FIG. 1B is a block diagram illustrating physical neural network 100 in more detail, according to example embodiments. As shown, physical neural network 100 may be composed of a plurality of layers 112. For example, as shown, physical neural network 100 may be composed of layer 112a, layer 112b, layer 112c, layer 112d, and layer 112n. In this particular example, layer 112a may be the first layer in physical neural network 100. As shown, layer 112a may receive, as input, data input 114 and parameters 116. Based on data input 114 and parameters 116, layer 112a may generate an output 118. Data input 114, parameters 116, and output 118 may be provided, as input, to the following layer, i.e., layer 112b. Based on data input 114, parameters 116, and output 118, layer 112b may generate output 120. Data input 114, parameters 116, and output 120 may be provided, as input, to the following layer, i.e., layer 112c. Such process may continue for each layer 112d to layer 112n to generate a final output 122.

As shown above, physical neural network 100 may illustrate a universal framework to directly train arbitrary, real physical systems to execute deep neural networks, using backpropagation. The trained hierarchical physical computations may be referred to as the physical neural networks (PNNs). A hybrid physical-digital algorithm, i.e., physics-aware training (PAT), allows for the efficient and accurate execution of a backpropagation algorithm on any sequence of physical input-output transformations, directly in situ. While PNNs are a radical departure from traditional hardware, they are easily integrated into modern machine learning. For example, PNNs can be seamlessly combined with conventional hardware and neural network methods via physical-digital hybrid architectures, in which conventional hardware learns to opportunistically cooperate with unconventional physical resources using PAT. Ultimately, PNNs provide a basis for hardware-physics-software codesign in artificial intelligence, routes to improving the energy efficiency and speed of machine learning by many orders of magnitude, and pathways to automatically designing complex functional devices, such as functional nanoparticles, robots, smart sensors, and the like.

To train parameters of physical neural network 100, PAT may be used. PAT is an algorithm that allows for a backpropagation algorithm for stochastic gradient descent (SGD) to be performed directly on any sequence of physical input-output transformations. In some embodiments, in the backpropagation algorithm, automatic differentiation may efficiently determine the gradient of a loss function with respect to trainable parameters. This makes the algorithm around N-times more efficient than finite-difference methods for gradient estimation (where N is the number of parameters). PAT may have some similarities to quantization-aware training algorithms used to train neural networks for low-precision hardware and feedback alignment. PAT can be seen as solving a problem analogous to the “simulation-reality gap” in robotics, which is increasingly addressed by hybrid physical-digital techniques.

As previously mentioned, physics-aware training (PAT) is a gradient-based learning algorithm. The algorithm may compute the gradients of the loss with respect to the parameters of the network. Since the loss may indicate how well the network is performing at its machine learning task, the gradients of the loss are subsequently used to update the parameters of the network. The gradients may computed efficiently via a backpropagation algorithm.

The backpropagation algorithm is commonly applied to neural networks composed of differentiable functions. It involves two key steps: a forward pass to compute the loss and a backward pass to compute the gradients with respect to the loss. The mathematical technique underpinning this algorithm may be referred to as reverse-mode automatic differentiation (autodiff). In some embodiments, each differentiable function in the network may be an autodiff function which may specify how signals propagate forward through the network and how error signals propagate backward. Given the constituent autodiff functions and a prescription for how these different functions are connected to each other, i.e., the network architecture, reverse-mode autodiff may be able to compute the desired gradients iteratively from the outputs towards the inputs and parameters (heuristically termed “backward”) in an efficient manner. For example, the output of a conventional deep neural network may be given by ƒ(ƒ( . . . ƒ(ƒ(x, θ¹), θ²) . . . , θ^{[N-1]}), θ^{N}). Here, ƒ may denote the constituent autodiff function. For example, ƒ may be given by ƒ(x, θ)=Relu(Wx+b), where the weight matrix W and the bias b may be representative of the parameters of a given layer and Relu may be the rectifying linear unit activation function (although other activation functions may be used). Given a prescription for how the forward and backward pass is performed for ƒ, the autograd algorithm may be able to compute the overall loss of the network.

In physics aware training, an alternative implementation of a conventional backpropagation algorithm may be used. For example, the present variant may employ autodiff functions that may utilize different functions for the forward and backward pass.

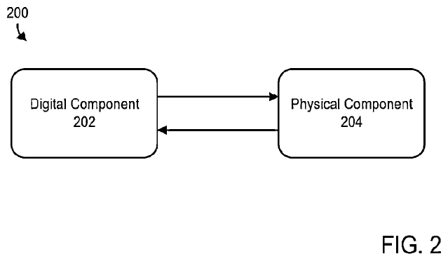

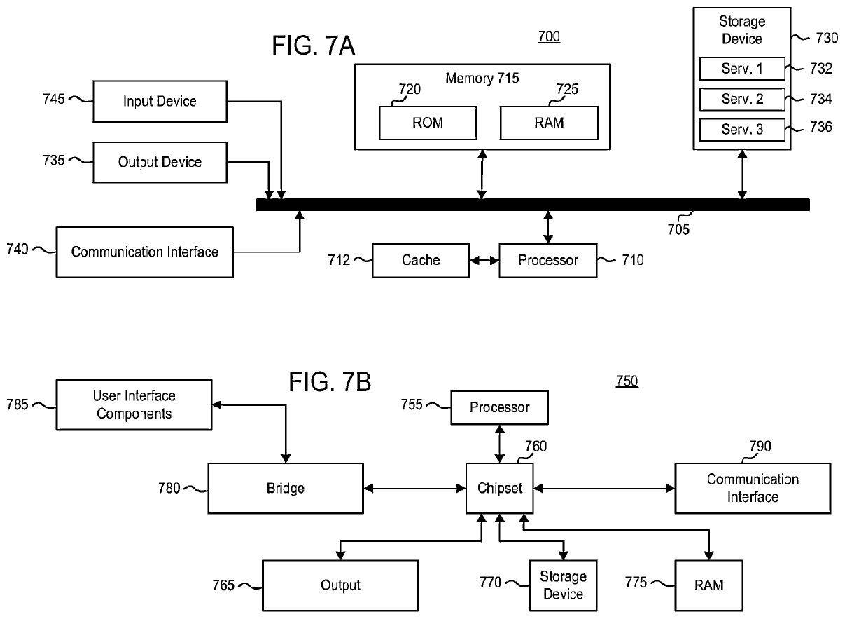

FIG. 2 is a block diagram illustrating a generic physical neural network system 200, according to example embodiments. As shown, physical neural network system 200 may include a digital component 202 and a physical component 204. Digital component 202 may be configured to generate the input data sets for physical component 204. Generally, digital component 202 may be representative of a computing system, such as that described below in conjunction with FIGS. 7A and 7B. Physical component 204 may be representative of a physical system, such as, but not limited to, a mechanical system, an optical system, an electronics system, or the like. More generally, physical component 204 may be configured to perform what would conventionally be a forward pass of a backpropagation process to generate an output. Digital component 202 may be configured to perform what would conventionally be the backward pass of the backpropagation process based on the generated output from the forward pass. The overall training process is further described below.

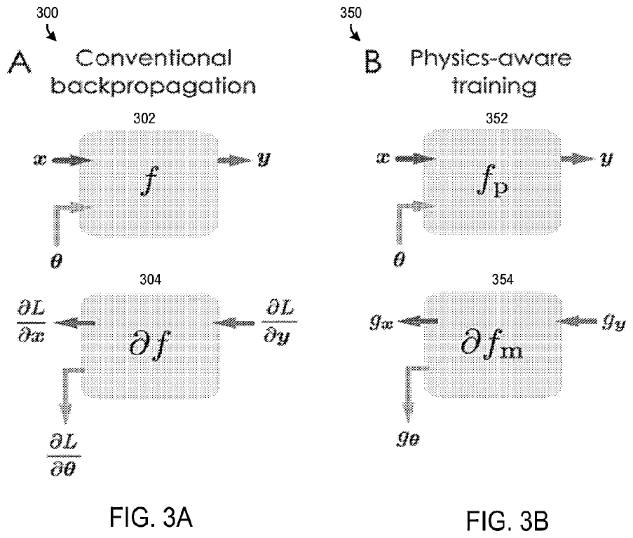

FIG. 3A is a block diagram illustrating a conventional backpropagation process 300, according to example embodiments. FIG. 3B is a block diagram illustrating the PAT backpropagation process 350, according to example embodiments.

As shown in FIG. 3A, the conventional backpropagation algorithm uses the same function for the forward pass 302 and the backward pass 304. Mathematically, forward pass 302, w n s t put and parameters into the output, is given by y=ƒ(x, θ), where x∈ [symbol] ⁿ is the input, θ∈ [symbol] ^{p} are the parameters, y∈ [symbol] ^{m} is the output of the map, and ƒ: [symbol] ⁿ× [symbol] ^{p}→ [symbol] ^{m} represents some general function that is an constituent operation which is applied in the overall neural network.

Backward pass 304, which maps the gradients with respect to the output into gradients with respect to the input and parameters, may be given by the following Jacobian-vector product:

$$\frac{\partial L}{\partial x} = {( \frac{\partial f}{\partial x} ( x , θ ) )}^{T} \frac{\partial L}{\partial y}$$

$$\frac{\partial L}{\partial θ} = {( \frac{\partial f}{\partial θ} ( x , θ ) )}^{T} \frac{\partial L}{\partial y}$$

where ∂L/∂y∈ [symbol] ^{m}, ∂L/∂x∈ [symbol] ⁿ, ∂L/∂θ∈ [symbol] ^{p} may represent the gradients of the loss with respect to the output, input, and parameters, respectively.

$$\frac{\partial L}{\partial x} = ( \frac{\partial f}{\partial x} ( x , θ ) )$$

∈ [symbol] ^{m×n} may denote the Jacobian matrix of the function ƒ with respect to x evaluated at (x, θ), i.e.,

$${( \frac{\partial f}{\partial x} ( x , θ ) )}_{ij} = \frac{\partial {f}_{i}}{\partial {x}_{j}} ( x , θ ) .$$

### Analogously,

$${( \frac{\partial f}{\partial θ} ( x , θ ) )}_{ij} = \frac{\partial {f}_{i}}{\partial {θ}_{j}} ( x , θ ) .$$

Though the conventional backpropagation algorithm described in FIG. 3A may work well for deep learning with digital computers—it cannot be naively applied to physical neural networks. This is because the analytic gradients of input-output transformations performed by physical systems (such as ∂ƒ/∂x(x, θ)) cannot be computed. The derivatives could be estimated via a finite-difference approach—requiring n+p number of calls to the physical system per backward pass. The resulting algorithm would be n+p times slower, which is significant for reasonably sized networks with thousands or millions of trainable parameters.

In contrast, FIG. 3B illustrates an alternative backpropagation algorithm that is suitable for arbitrary real physical input-output transformations. PAT backpropagation process 350 may employ autodiff functions that may utilize different functions for forward pass 352 and backward pass 354. As shown schematically in FIG. 3B, a non-differentiable transformation of the physical system (ƒₚ) may be used in forward pass 352 and a differentiable digital model of the physical transformation (ƒₘ) may be used in backward pass 354. Because physics-aware training is a backpropagation algorithm, which may efficiently compute gradients in an iterative manner, it is able to train the physical system efficiently. Moreover, as it is formulated in the same paradigm of reverse-mode automatic differentiation, a user can define these custom autodiff functions in any conventional machine learning library (such as PyTorch) to design and train physical neural networks with the same workflow adopted for conventional neural networks.

For a constituent physical transformation in the overall physical neural network, the forward pass operation of this constituent may be given by y=ƒₚ(x, θ). As a different function is used in backward pass 354 than forward pass 352, the autodiff function may no longer be able to backpropagate the gradients at the output layer to the exact gradients at the input layer. Instead, it may strive to approximate the backpropagation of the exact gradients. Thus, backward pass 354 may be given by:

$${x}_{} = {( \frac{\partial {f}_{m}}{\partial x} ( x , θ ) )}^{T} {y}_{}$$

$${θ}_{} = {( \frac{\partial {f}_{m}}{\partial θ} ( x , θ ) )}^{T} {y}_{}$$

where g_{y}, gₓ, and g_{θ} may be estimators of the gradients ∂L/∂y, ∂L/∂x, and ∂L/∂θ, respectively.

In other words, in PAT training, backward pass 354 may be estimated using a differentiable digital model, i.e., ƒₘ(x, θ), while forward pass 352 may be implemented by the physical system, i.e., ƒₚ(x, θ).

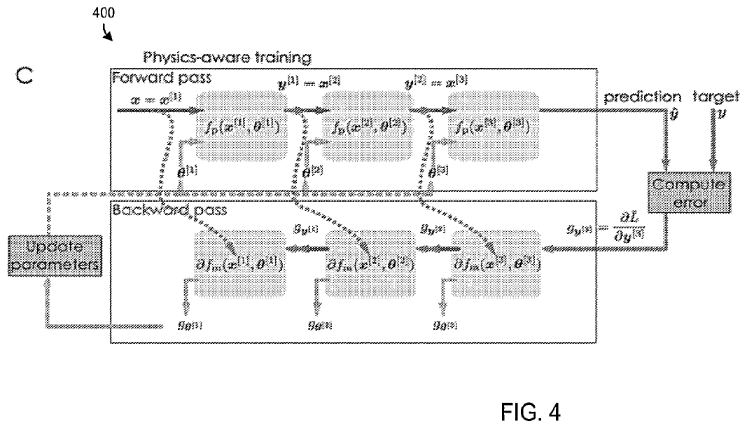

FIG. 4 is a block diagram illustrating a detailed physics-aware training process 400, according to example embodiments. As shown, FIG. 4 may illustrate a full training loop for physics-aware training. Such discussion may utilize a feedforward PNN architecture as it is the PNN counterpart of the canonical feedforward neural network (multilayer perceptrons) in deep learning. Physics-aware training is specifically designed to avoid the pitfalls of both the ideal backpropagation algorithm and in silico training. It is a hybrid algorithm involving computation in both the physical and digital domain.

More specifically, the physical system is used to perform the forward pass, which alleviates the burden of having the differential digital models be exceptionally accurate (as in in silico training). The differentiable digital model may only be utilized in the backward pass to complement parts of the training loop that the physical system cannot perform. Physics-aware training can be formalized by the use of custom constituent autodiff-functions in an overall network architecture. In the case of the feedforward PNN, the autodiff algorithm with these custom functions may result in and may simplify to the following training loop:

$$Perform Forward Pass : {x}^{[ L + 1 ]} = {y}^{[ l ]} = {f}_{p} ( {x}^{[ l ]} , {θ}^{[ l ]} )$$

$$Compute Error Vector : {{y}^{[ N ]}}_{} = \frac{\partial L}{\partial {y}^{[ N ]}} = \frac{\partial ℓ}{\partial {y}^{[ N ]}} ( {y}^{[ N ]} , y )$$

$$Perform Backward Pass : {{y}^{[ l - 1 ]}}_{} = {( \frac{\partial {f}_{m}}{\partial x} ( {x}^{[ l ]} , {θ}^{[ l ]} ) )}^{T} {{y}^{[ l ]}}_{}$$

$${{θ}^{[ l ]}}_{} = {( \frac{\partial {f}_{m}}{\partial x} ( {x}^{[ l ]} , {θ}^{[ l ]} ) )}^{T} {{y}^{[ l ]}}_{}$$

$$Update parameters : {θ}^{[ l ]} \to {θ}^{[ l ]} - η \frac{1}{{N}_{dαtα}} {\sum }_{k} {{θ}^{[ l ]}}_{k}^{}$$

where g_{θ}_{^{[l]}} and g_{y}_{^{[l]}} are estimators of the “exact” gradients ∂L/∂θ^{[l]}, ∂L/∂y^{[l]}, respectively. Thus, the angle between the estimated gradient vector and the exact gradient vector ∠(∂L/∂θ^{[l]}, [symbol] _{θ}_{^{[l]}}) conceptually characterized the performance of physics aware training. In physics aware training, the error vector may exact

$$( {{y}^{[ N ]}}_{} = \frac{\partial L}{\partial {y}^{[ N ]}} )$$

as the forward pass may be performed by the physical system. The error vector may then be backpropagated via the backward pass, which may involve Jacobian matrices of the differential digital model evaluated at the “correct” inputs (x^{[l]} instead of the predicted \tilde{x}^{[l]}) at each layer. Thus, in addition to utilizing the output of the PNN (y^{[N]}) via physical computations in the forward pass, intermediate outputs (y^{[l]}) may also be utilized to facilitate the computation of accurate gradients in physics-aware training.

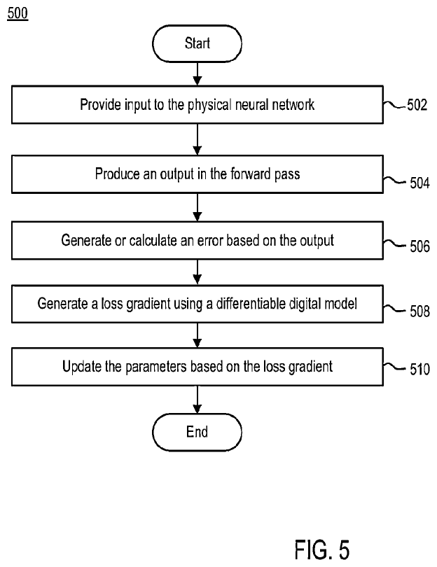

FIG. 5 is a flow diagram illustrating a method 500 of training a physical neural network (PNN) using physics-aware training, according to example embodiments. Method 500 may begin as step 502.

As step 502, a computing system may provide input to the PNN. In some embodiments, the input may include training data and trainable parameters. In some embodiments, the training data and the trainable parameters may be encoded prior to input to the PNN. Using a specific example, input data and parameters may be encoded into a time-dependent force applied to a suspended metal plate.

At step 504, PNN may use its transformations to produce an output in the forward pass. For example, as recited above, in the forward pass, PNN may perform: x^{[l+1]}=y^{[l]}=ƒₚ(x^{[l]}, θ^{[l]}) to generate an output in the forward pass.

At step 506, a computing system may generate or calculate an error. For example, the computing system may compare the actual physical output to a canonical or expected physical output. The difference between the actual physical output and the canonical or expected physical output may represent the error. In some embodiments, the error vector may be generated by:

$${{y}^{[ N ]}}_{} = \frac{\partial L}{\partial {y}^{[ N ]}} = \frac{\partial ℓ}{\partial {y}^{[ N ]}} ( {y}^{[ N ]} , y ) .$$

At step 508, the computing system may generate a loss gradient using a differentiable digital model. For example, using a differentiable digital model to estimate the gradients of PNN, the computing system may generate the gradient of the loss with respect to the controllable parameters. A backward pass may be performed using:

$${{y}^{[ l - 1 ]}}_{} = {( \frac{\partial {f}_{m}}{\partial x} ( {x}^{[ l ]} , {θ}^{[ l ]} ) )}^{T} {{y}^{[ l ]}}_{}$$

$${{θ}^{[ l ]}}_{} = {( \frac{\partial {f}_{m}}{\partial θ} ( {x}^{[ l ]} , {θ}^{[ l ]} ) )}^{T} {{y}^{[ l ]}}_{}$$

At step 510, the computing system may update the parameters. For example, computing system may update the parameters of the system based on the estimated gradient.

Such process may continue until conversion.

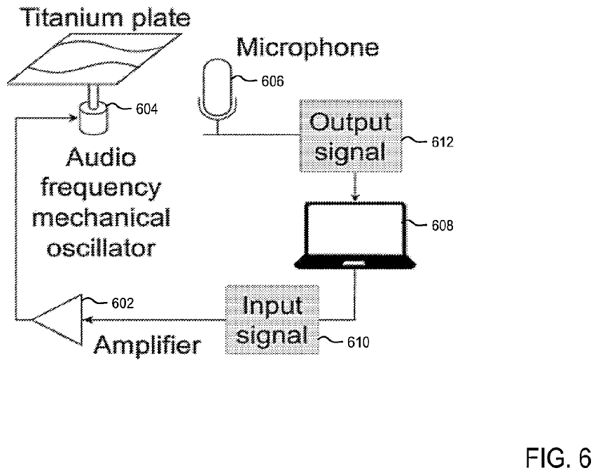

FIG. 6 is a block diagram illustrating an example physical neural network (PNN) 600, according to example embodiments. As shown, PNN 600 may be representative of an oscillating plate experimental setup. For example, PNN 600 may include an audio amplifier 602, a commercially available full-range speaker 604, a microphone 606, and a computer 608 controlling the setup. Computer 608 may provide an input signal 610 to amplifier 602. In some embodiments, input signal 610 may include encoded physical inputs in the time domain. For example, computer 608 may encode inputs in a time-series of rectangular pulses that may be transformed into an analog signal by a digital-to-analog convertor of computer 608.

Amplifier 602 may be configured to amplify the input signal received from computer 608 and apply the amplified input signal to a mechanical oscillator realized by the voice coil of an acoustic speaker. For example, the speaker may be used to drive mechanical oscillations of a titanium plate that may be mounted on the speaker's voice coil.

Microphone 606 may be configured to record the sound produced by the oscillating plate. In some embodiments, the sound recorded by microphone 606 may be converted back to digital. The recorded sound may be representative of an output signal 612 provided back to computer 608. Computer 608 may be configured to compare output signal 612 to an expected output signal to generate the error. Computer 608 may further be configured to evaluate the loss gradient with respect to the controllable parameters using the digital model. Based on the generated gradient, computer 608 may update or change the parameters passed to amplifier 602.

FIG. 7A illustrates a system bus architecture of computing system 700, according to example embodiments. System 700 may be representative of at least a portion of digital component 202. One or more components of system 700 may be in electrical communication with each other using a bus 705. System 700 may include a processing unit (CPU or processor) 710 and a system bus 705 that couples various system components including the system memory 715, such as read only memory (ROM) 720 and random access memory (RAM) 725, to processor 710. System 700 may include a cache of high-speed memory connected directly with, in close proximity to, or integrated as part of processor 710. System 700 may copy data from memory 715 and/or storage device 730 to cache 712 for quick access by processor 710. In this way, cache 712 may provide a performance boost that avoids processor 710 delays while waiting for data. These and other modules may control or be configured to control processor 710 to perform various actions. Other system memory 715 may be available for use as well. Memory 715 may include multiple different types of memory with different performance characteristics. Processor 710 may include any general purpose processor and a hardware module or software module, such as service 1 732, service 2 734, and service 3 736 stored in storage device 730, configured to control processor 710 as well as a special-purpose processor where software instructions are incorporated into the actual processor design. Processor 710 may essentially be a completely self-contained computing system, containing multiple cores or processors, a bus, memory controller, cache, etc. A multi-core processor may be symmetric or asymmetric.

To enable user interaction with the computing system 700, an input device 745 may represent any number of input mechanisms, such as a microphone for speech, a touch-sensitive screen for gesture or graphical input, keyboard, mouse, motion input, speech and so forth. An output device 735 may also be one or more of a number of output mechanisms known to those of skill in the art. In some instances, multimodal systems may enable a user to provide multiple types of input to communicate with computing system 700. Communications interface 740 may generally govern and manage the user input and system output. There is no restriction on operating on any particular hardware arrangement and therefore the basic features here may easily be substituted for improved hardware or firmware arrangements as they are developed.

Storage device 730 may be a non-volatile memory and may be a hard disk or other types of computer readable media which may store data that are accessible by a computer, such as magnetic cassettes, flash memory cards, solid state memory devices, digital versatile disks, cartridges, random access memories (RAMs) 725, read only memory (ROM) 720, and hybrids thereof.

Storage device 730 may include services 732, 734, and 736 for controlling the processor 710. Other hardware or software modules are contemplated. Storage device 730 may be connected to system bus 705. In one aspect, a hardware module that performs a particular function may include the software component stored in a computer-readable medium in connection with the necessary hardware components, such as processor 710, bus 705, output device 735 (e.g., display), and so forth, to carry out the function.

FIG. 7B illustrates a computer system 750 having a chipset architecture that may represent at least a portion of digital component 202. Computer system 750 may be an example of computer hardware, software, and firmware that may be used to implement the disclosed technology. System 750 may include a processor 755, representative of any number of physically and/or logically distinct resources capable of executing software, firmware, and hardware configured to perform identified computations. Processor 755 may communicate with a chipset 760 that may control input to and output from processor 755. In this example, chipset 760 outputs information to output 765, such as a display, and may read and write information to storage device 770, which may include magnetic media, and solid state media, for example. Chipset 760 may also read data from and write data to storage device 775 (e.g., RAM). A bridge 780 for interfacing with a variety of user interface components 785 may be provided for interfacing with chipset 760. Such user interface components 785 may include a keyboard, a microphone, touch detection and processing circuitry, a pointing device, such as a mouse, and so on. In general, inputs to system 750 may come from any of a variety of sources, machine generated and/or human generated.

Chipset 760 may also interface with one or more communication interfaces 790 that may have different physical interfaces. Such communication interfaces may include interfaces for wired and wireless local area networks, for broadband wireless networks, as well as personal area networks. Some applications of the methods for generating, displaying, and using the GUI disclosed herein may include receiving ordered datasets over the physical interface or be generated by the machine itself by processor 755 analyzing data stored in storage device 770 or storage device 775. Further, the machine may receive inputs from a user through user interface components 785 and execute appropriate functions, such as browsing functions by interpreting these inputs using processor 755.

It may be appreciated that example systems 700 and 750 may have more than one processor 710 or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A physical neural network system, comprising:
a physical component; and
a digital component comprising a computing system, wherein the physical component and the digital component work in conjunction to execute a physics aware training process, comprising:
generating, by the digital component, an input data set for input to the physical component;
applying, by the physical component, one or more transformations to the input data set to generate an output for a forward pass of the physics aware training process;
based on the generated output, comparing, by the digital component, the generated output to a canonical output to determine an error;
generating, by the digital component, a loss gradient using a differentiable digital model for a backward pass of the physics aware training process; and
updating, by the digital component, training parameters for subsequent input to the physical component based on the loss gradient.

2. The physical neural network system of claim 1, wherein applying, by the physical component, the one or more transformations to the input data set to generate the output for the forward pass of the physics aware training process comprises:
employing at least one non-differentiable transformation function to the input data set in the forward pass.

3. The physical neural network system of claim 2, wherein generating, by the digital component, the loss gradient using the differentiable digital model for the backward pass of the physics aware training process comprises:
approximating the loss gradient using the differentiable digital model, wherein the differentiable digital model in the backward pass is different from the at least one non-differentiable transformation function of the forward pass.

4. The physical neural network system of claim 1, wherein the physical component comprises a first layer and a second layer.

5. The physical neural network system of claim 4, wherein applying, by the physical component, the one or more transformations to generate the output for the forward pass of the physics aware training process comprises:
applying, by the first layer, a first transformation to the input data set to generate an intermediary output; and
applying, by the second layer, a second transformation to the input data set and the intermediary output to generate the output.

6. The physical neural network system of claim 1, wherein generating, by the digital component, the input data set for input to the physical component comprises:
encoding input data and initial parameters to generate the input data set.

7. The physical neural network system of claim 6, further comprising:
generating, by the digital component, an updated input data set by encoding the input data and the updated training parameters.

8. A method of training a physical neural network, comprising:
generating, by a digital component of the physical neural network, an input data set for input to the physical component;
applying, by a physical component of the physical neural network, one or more transformations to the input data set to generate an output for a forward pass of the training;
based on the generated output, comparing, by the digital component, the generated output to a canonical output to determine an error;
generating, by the digital component, a loss gradient using a differentiable digital model for a backward pass of the training; and
updating, by the digital component, training parameters for subsequent input to the physical component based on the loss gradient.

9. The method of claim 8, wherein applying, by the physical component, the one or more transformations to the input data set to generate the output for the forward pass of the training comprises:
employing at least one non-differentiable transformation function to the input data set in the forward pass.

10. The method of claim 9, wherein generating, by the digital component, the loss gradient using the differentiable digital model for the backward pass of the training comprises:
approximating the loss gradient using the differentiable digital model, wherein the differentiable digital model in the backward pass is different from the at least one non-differentiable transformation function of the forward pass.

11. The method of claim 8, wherein the physical component comprises a first layer and a second layer.

12. The method of claim 11, wherein applying, by the physical component, the one or more transformations to generate the output for the forward pass of the training comprises:
applying, by the first layer, a first transformation to the input data set to generate an intermediary output; and
applying, by the second layer, a second transformation to the input data set and the intermediary output to generate the output.

13. The method of claim 8, wherein generating, by the digital component, the input data set for input to the physical component comprises:
encoding input data and initial parameters to generate the input data set.

14. The method of claim 13, further comprising:
generating, by the digital component, an updated input data set by encoding the input data and the updated training parameters.

15. A non-transitory computer readable medium comprising one or more sequences of instructions, which, when executed by one or more processors, causes a computing system to perform operations, comprising:
generating an input data set for input to a physical component of a physical neural network;
causing the physical component of the physical neural network to apply one or more transformations to the input data set to generate an output for a forward pass of a training process;
based on the generated output, comparing the generated output to a canonical output to determine an error;
generating a loss gradient using a differentiable digital model for a backward pass of the training process; and
updating training parameters for subsequent input to the physical component based on the loss gradient.

16. The non-transitory computer readable medium of claim 15, wherein applying, by the physical component, the one or more transformations to the input data set to generate the output for the forward pass of the training process comprises:
employing at least one non-differentiable transformation function to the input data set in the forward pass.

17. The non-transitory computer readable medium of claim 16, wherein generating the loss gradient using the differentiable digital model for the backward pass of the training process comprises:
approximating the loss gradient using the differentiable digital model, wherein the differentiable digital model in the backward pass is different from the at least one non-differentiable transformation function of the forward pass.

18. The non-transitory computer readable medium of claim 15, wherein the physical component comprises a first layer and a second layer.

19. The non-transitory computer readable medium of claim 18, wherein causing the physical component to apply the one or more transformations to generate the output for the forward pass of the training process comprises:
causing the first layer to apply a first transformation to the input data set to generate an intermediary output; and
causing the second layer to apply a second transformation to the input data set and the intermediary output to generate the output.

20. The non-transitory computer readable medium of claim 15, wherein generating the input data set for input to the physical component comprises:
encoding input data and initial parameters to generate the input data set.
